(19) United States
(12) Patent Application Publication
(10) Pub. No.: US 2012/0000118 A1
(43) Pub. Date: Jan. 5, 2012

(54) LOW-MOLECULAR WEIGHT POLYISOBUTYL-SUBSTITUTED AMINES AS DISPERSANT BOOSTERS

(75) Inventors: Arno LANGE, Bad Duerkheim (DE); Peter Schreyer, Weinheim (DE); Robert Stuart Grace, Sherbourne St. John (GB)

(73) Assignee: BASF SE, Ludwigshafen (DE)

(21) Appl. No.: 13/150,776

(22) Filed: Jun. 1, 2011

Related U.S. Application Data
(60) Provisional application No. 61/350,048, filed on Jun. 1, 2010.

(51) Int. Cl. C10L 1/233; C10L 1/232; C07D 207/30; C07C 215/08; C07D 211/12; C07D 265/30; C10L 1/222; C07C 211/03

(52) U.S. Cl. 44/334; 44/412; 44/434; 44/339; 44/340; 564/463; 564/503; 546/184; 544/106; 548/579

(57) ABSTRACT

A fuel additive composition comprising (A) polyisobutyl-based nitrogen-containing dispersants with M_{N} of the polyisobutyl group of from 650 to 1800 Dalton, (B) carrier oils substantially free of nitrogen and (C) polyisobutyl-based dispersant boosters with M_{N} of the polyisobutyl group of from 200 to 650 Dalton, with the proviso that the difference between the M_{N} of the polyisobutyl group of component (A) and the M_{N} of the polyisobutyl group of component (C) is more than 100 Dalton. Said component (C) is especially useful as an intake valve clean-up booster in gasoline-operated port fuel injection internal combustion engines.

The present invention relates to a novel fuel additive composition comprising (A) nitrogen-containing dispersants selected from polyisobutyl monoamines, polyisobutyl polyamines, Mannich adducts of polyisobutylphenols, aldehyds and monoamines and Mannich adducts of polyisobutylphenols, aldehyds and polyamines, (B) carrier oils which are substantially free of nitrogen and which are selected from synthetic carrier oils and mineral carrier oils, and (C) dispersant boosters selected from low-molecular weight polyisobutyl monoamines, polyisobutyl polyamines, Mannich adducts of polyisobutylphenols, aldehyds and monoamines and Mannich adducts of polyisobutylphenols, aldehyds and polyamines. Furthermore, the present invention relates to a gasoline fuel composition comprising a minor amount of the said fuel additive composition. Furthermore, the present invention relates to the use of such low-molecular weight polyisobutyl-substituted amines as dispersant boosters in internal combustion engines operated with gasoline containing the above detergents and the above carrier oils.

### TECHNICAL BACKGROUND

Carburettors and inlet systems of automobile engines, and also injection systems for fuel proportioning, are subjected to increasing load due to contamination caused by dust particles from the air, unburned hydrocarbon residues from the combustion chamber and crankcase ventilation, and exhaust gas recycle passed to the intake system.

These residues shift the air-to-fuel ratio during idling and in the lower partial load region, so that the mixture becomes richer and combustion less complete and consequently the content of unburned or partly burned hydrocarbons in the exhaust gas increases and the gasoline consumption rises.

It is known that these drawbacks may be avoided through the use of fuel additives for cleaning the valves and carburettors or injection systems of Otto engines (cf. e.g.: M. Rossenbeck in “Katalysatoren, Tenside, Mineralöladditive”, edited by J. Falbe, U. Hasserodt, page 223, G. Thieme Verlag, Stuttgart 1978).

For trouble-free running, modern Otto engines require automotive fuels having a complex set of properties which can only be guaranteed when use is made of appropriate gasoline additives. Such fuels usually consist of a complex mixture of chemical compounds and are characterized by physical parameters.

Fuel additives are i.a. used in order to avoid formation of deposits in the intake system and the intake valves of engines (keep-clean effect). On the other hand, fuel additives may be used in order to remove deposits already formed at the valves and in the intake system (clean-up effect).

Aliphatic primary, secondary and tertiary monoamines with C₁-C₂₀-alkyl residues or C₃-C₂₀-cycloalkyl residues are known as dispersant additives in gasoline fuels, preferably in combination with Mannich-type dispersant additives, from WO 04/050806. The said monoamines can be used in gasoline fuels together with other dispersants additives, such as polyisobutyl monoamines or polyisobutyl polyamines based on polyisobutene with a number average molecular weight of from 600 to 5000, and with polyether carrier oils, such as tridecanol butoxylate or isotridecanol butoxylate. The use of the said monoamines results in a reduction of injector nozzle fouling in direct injection spark ignition engines.

WO 03/076554 relates to the use of hydrocarbyl amines wherein the hydrocarbyl moiety has a number average molecular weight in the range of from 140 to 255 for reducing injector nozzle fouling in direct injection spark ignition engines, either for “keep clean” or for “clean-up” purposes of such engines. In Fuel D of the examples of WO 03/076554, a gasoline fuel was prepared by “dosing into the base fuel 645 ppmw of a commercial additive package ex BASF A.G., containing polyisobutyl monoamine (PIBA), in which the polyisobutylene (PIB) chain has a number average molecular weight (M_{N}) of approximately 1000, a polyether carrier fluid and an antioxidant, with further inclusion of 50 ppmw dodecylamine”. Fuel D was subjected to a clean-up test determining the average injector diameter reduction after running a direct injection spark ignition engine with this Fuel.

WO 90/10051 relates to a gasoline fuel composition containing an intake valve deposit control additive formulation comprising (1) long-chain primary amines exhibiting typically C₆-C₄₀ aliphatic radicals as substituents, e.g. decyl amine, dodecyl amine (lauryl amine), or tallow amines containing tetradecyl amine, hexadecyl amine, octadecyl amine and octadecenyl amine (oleyl amine), in combination with (2) fuel dispersants selected from polyalkylamines (such polyisobutyl amine) and Mannich bases, and with (3) fluidizer oils such as refined napthenic lubricating oil or a polyolefin like polypropylene or polybutylene.

US 2007/0094922 A1 relates to polyalkene amines such as polyisobutyl monoamines with improved applicational properties for use as additives in fuel or lubricant compositions. Suitable polyisobutyl monoamines are those derived from highly reactive polyisobutenes available from BASF AG under the Glissopal® brands, in particular “Glissopal 1000 (Mn=1000), Glissopal V 33 (Mn=550) and Glissopal 2300 (Mn=2300) and mixtures thereof”. The polyalkene amines such as polyisobutyl monoamines disclosed in US 2007/0094922 A1 can be used together with mineral carrier oils or synthetic carrier oils.

U.S. Pat. No. 3,898,056 discloses a mixture of high and low molecular weight hydrocarbyl amines for use in the automotive fuel additive area. The high molecular weight hydrocarbyl amines contain hydrocarbyl groups having a molecular weight between about 1900 and 5000; these amines may be conveniently prepared by reacting a corresponding hydrocarbyl halide with a monoamine or polyamine. The low molecular weight hydrocarbyl amines contain hydrocarbyl groups having a molecular weight between about 300 and 600; these amines may be also conveniently prepared by reacting a corresponding hydrocarbyl halide with a monoamine or polyamine. Examples for such high and low molecular weight hydrocarbyl amines are prepared from corresponding polyisobutylenes. The high and low molecular weight hydrocarbyl amines disclosed in U.S. Pat. No. 3,898,056 can be used together with fuel soluble carrier oils such as nonvolatile lubricating mineral oils or polyalkoxy polyols.

The interrelationship between gasoline fuels and appropriate fuel additives in fuel compositions may still be unsatisfactory as regards their intake valve clean-up performance. It is, therefore, an object of the present invention to provide improved fuel additive formulations which allow an efficient control of deposits formed in the engine, especially an improved intake valve clean-up performance.

### BRIEF DESCRIPTION OF THE INVENTION

It has now been observed that a fuel additive composition comprising:

(A) at least one nitrogen-containing dispersant selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 650 to 1800 Dalton,

(B) at least one carrier oil which is substantially free of nitrogen, selected from synthetic carrier oils and mineral carrier oils, and

(C) at least one dispersant booster selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 200 to 650 Dalton,

with the proviso that the difference between the M_{N} of the polyisobutyl group of component (A) and the M_{N} of the polyisobutyl group of component (C) is more than 100 Dalton, preferably is more than 250 Dalton, more preferably is in the range of from more than 100 to 900 Dalton and most preferably is in the range of from more than 250 to 600 Dalton,

improves the intake valve clean-up performance of gasoline fuels significantly. Therefore, the said fuel additive composition is a first subject matter of the instant invention.

A second subject matter of the instant invention is a fuel composition comprising a major amount of a liquid fuel in gasoline boiling range and a minor amount of the above fuel additive composition.

A third subject matter of the instant invention is the use of a polyisobutyl monoamine, a polyisobutyl polyamine, a Mannich adduct of a polyisobutylphenol, an aldehyd and a monoamine or a Mannich adduct of a polyisobutylphenol, an aldehyd and a polyamine (C), each with a number average molecular weight M_{N} of the polyisobutyl group of from 200 to 650 Dalton, as set out in Claim 1, as a dispersant booster in internal combustion engines operated with a liquid fuel in the gasoline boiling range containing minor amounts of (A) at least one nitrogen-containing dispersant selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 650 to 1800 Dalton, and (B) at least one carrier oil which is substantially free of nitrogen, selected from synthetic carrier oils and mineral carrier oils.

### DETAILS DESCRIPTION OF THE INVENTION AND PREFERRED EMBODIMENTS

The nitrogen-containing dispersant (Component A)

The polyisobutenes which are suitable for preparing the polyisobutyl monoamines, polyisobutenyl polyamines and polyisobutyl-substituted Mannich adducts used in the present invention include polyisobutenes which comprise at least about 20 mol-%, preferably at least 50 mol-%, more preferably at least 70 mol-%, most preferably at least 80 mol-%, of the more reactive methylvinylidene isomer (i.e. with the terminal vinylidene double bond). Suitable polyisobutenes include those prepared using BF₃ catalysts. The preparation of such polyisobutenes in which the methylvinylidene isomer comprises a high percentage of the total composition is for example described in U.S. Pat. No. 4,152,499 and U.S. Pat. No. 4,605,808, starting either from pure isobutene or from technical C₄ streams containing high percentages of isobutene such as raffinate I.

Examples of suitable polyisobutenes having a high methylvinylidene content include products like Ultravis® 30, a polyisobutene having a number average molecular weight of about 1300 and a methylvinylidene content of about 74 mol-%, and Ultravis® 10, a 950 molecular weight polyisobutene having a methylvinylidene content of about 76 mol-%, both of British Petroleum. Another example of a suitable polyisobutene having a number average molecular weight of about 1000 and a high methylvinyliden content is Glissopal® 1000, available from BASF SE.

In most instances, the polyisobutene precursors are not a pure single product, but rather a mixture of compounds having a number average molecular weight in the above range. Usually, the range of molecular weights distribution will be relatively narrow having a maximum near the indicated molecular weight.

The amine component of the polyisobutyl monoamines or polyisobutyl polyamines, respectively, may be derived from ammonia, a monoamine or a polyamine.

The monoamine or polyamine component comprises amines having from 1 to about 12 amine nitrogen atoms and from 1 to 40 carbon atoms. The carbon to nitrogen ratio may be between about 1:1 and about 10:1. Generally, the monoamine will contain from 1 to about 40 carbon atoms and the polyamine will contain from 2 to about 12 amine nitrogen atoms and from 2 to about 40 carbon atoms.

The amine component may be a pure single product or a mixture of compounds having a major quantity of the designated amine.

When the amine component is a polyamine, it will preferably be a polyalkylene polyamine, including alkylene diamine. Preferably, the alkylene group will contain from 2 to 6 carbon atoms, more preferably from 2, 3 or 4 carbon atoms. Examples of such polyamines include ethylene diamine, diethylene triamine, triethylene tetramine, tetraethylene pentamine and pentaethylene hexamine. Preferred polyamines are ethylene diamine and diethylene triamine.

Particularly preferred polyisobutyl polyamines include polyisobutyl ethylene diamine and polyisobutyl diethylene triamine. The polyisobutyl group is substantially saturated.

The polyisobutyl monoamines or polyisobutyl polyamines employed in the fuel additive composition of the instant invention are prepared by conventional procedures known in the art, especially by hydroformylation and subsequent reductive amination of corresponding highly reactive polyisobutenes, as described in EP-A 0 244 616. In more detail, highly reactive polyisobutenes having a high content of terminal vinylidene double bonds, especially at least 70 mol-%, more preferably at least 80 mol-%, of terminal vinylidene double bonds, are reacted with carbon monoxide and hydrogen in the presence of a hydroformylation catalyst, e.g. a suitable rhodium or cobalt catalyst, and preferably in an inert solvent such as a hydrocarbon solvent at a temperature in the range of typically from 80° C. to 200° C. and CO/H₂-pressures up to 600 bar. Thereafter, the oxo intermediate obtained is subjected to an reductive amination reaction in the presence of hydrogen, a suitable nitrogen compound, a suitable catalyst, e.g. Raney nickel or Raney cobalt, and preferably in an inert solvent such as a hydrocarbon solvent or an alcohol solvent at a temperature in the range of typically from 80° C. to 200° C. and H₂-pressures of from 80 to 300 bar.

The amine portion of the molecule may carry one or more substituents. Thus, the carbon and/or, in particular, the nitrogen atoms of the amine may carry substituents selected from hydrocarbyl groups of from 1 to about 10 carbon atoms, acyl groups of from 2 to about 10 carbon atoms, and monoketo, monohydroxy, mononitro, monocyano, lower alkyl and lower alkoxy derivatives thereof. “Lower” as used herein means a group containing from 1 to about 6 carbon atoms. At least one of the hydrogen atoms on one of the basic nitrogen atoms of the polyamine may not be substituted so that at least one of the basic nitrogen atoms of the polyamine is a primary or secondary amino nitrogen atom.

A polyamine finding use within the scope of the present invention as amine component for the polyisobutyl polyamines may be a polyalkylene polyamine, including substituted polyamines, e.g., alkyl and hydroxyalkyl-substituted polyalkylene polyamine. Among the polyalkylene polyamines, those containing 2 to 12 amino nitrogen atoms and 2 to 24 carbon atoms should be mentioned, in particular C₂-C₃ alkylene polyamines. Preferably, the alkylene group contains from 2 to 6 carbon atoms, there being preferably from 2 to 3 carbon atoms between the nitrogen atoms. Such groups are exemplified by ethylene, 1,2-propylene, 2,2-dimethylpropylene, trimethylene, 1,3-(2-hydroxy)-propylene.

Examples of such polyamines include ethylene diamine, diethylene triamine, di(trimethylene) triamine, 1,2-propylene diamine, 1,3-propylene diamine, dipropylene triamine, triethylene tetraamine, tripropylene tetraamine, tetraethylene pentamine, pentaethylene hexamine, hexamethylene diamine, and 3-(N,N-dimethylamino) propylamine. Such amines encompass isomers such as branched-chain polyamines and previously-mentioned substituted polyamines, including hydroxy- and hydrocarbyl-substituted polyamines.

The amine component for the polyisobutyl monoamines or polyisobutyl polyamines also may be derived from heterocyclic polyamines, heterocyclic substituted amines and substituted heterocyclic compounds, wherein the heterocycle comprises one or more five- to six-membered rings containing oxygen and/or nitrogen. Such heterocyclic rings may be saturated or unsaturated and substituted with groups as defined above.

As examples of heterocyclic compounds there may be mentioned 2-methylpiperazine, N-(2-hydroxyethyl)-piperazine, 1,2-bis-(N-piperazinyl)ethane, N,N′-bis(N-piperazinyl)piperazine, 2-methylimidazoline, 3-aminopiperidine, 3-aminopyridine, N-(3-aminopropyl)-morpholine, N-(beta-aminoethyl)piperazine, N-(betaaminoethyl)piperidine, 3-amino-N-ethylpiperidine, N-(betaaminoethyl) morpholine, N,N′-di(beta-aminoethyl)piperazine, N,N′-di(beta-aminoethyl)imidazolidone-2,1,3-dimethyl-5(beta-aminoethyl)hexahydrotriazine, N-(betaaminoethyl)-hexahydrotriazine, 5-(beta-aminoethyl)-1,3,5-dioxazine.

Alternatively, the amine component for the polyisobutyl monoamines may be derived from a monoamine having the formula HNR¹R² wherein R¹ and R² are independently selected from the group consisting of hydrogen and hydrocarbyl of 1 to about 20 carbon atoms and, when taken together, R¹ and R² may form one or more five- or six-membered rings containing up to about 20 carbon atoms. Preferably, R¹ is hydrogen and R² is a hydrocarbyl group having 1 to about 10 carbon atoms. More preferably, R¹ and R² are hydrogen. The hydrocarbyl groups may be straight-chain or branched and may be aliphatic, alicyclic, aromatic or combinations thereof. The hydrocarbyl groups may also contain one or more oxygen atoms.

Typical primary amines are exemplified by N-methylamine, N-ethylamine, N-n-propyl-amine, N-isopropylamine, N-n-butylamine, N-isobutylamine, N-sec.-butylamine, N-tert.-butylamine, N-n-pentylamine, N-cyclopentylamine, N-n-hexylamine, N-cyclohexylamine, N-octylamine, N-decylamine, N-dodecylamine, N-octadecylamine, N-benzylamine, N-(2-phenylethyl)amine, 2-aminoethanol, 3-amino-1-proponal, 2-(2-aminoethoxy)ethanol, N-(2-methoxyethyl)amine, N-(2-ethoxyethyl)amine, and the like. Preferred primary amines are N-methylamine, N-ethylamine and N-n-propylamine.

Typical secondary amines include N,N-dimethylamine, N,N-diethylamine, N,N-di-n-propylamine, N,N-diisopropylamine, N,N-di-n-butylamine, N,N-di-sec-butylamine, N,N-di-n-pentylamine, N,N-di-n-hexylamine, N,N-dicyclohexylamine, N,N-dioctylamine, N-ethyl-N-methylamine, N-methyl-N-n-propylamine, N-n-butyl-N-methylamine, N-methyl-N-octylamine, N-ethyl-N-isopropylamine, N-ethyl-N-octylamine, N,N-di-(2-hydroxyethyl)amine, N,N-di(3-hydroxypropyl)amine, N,N-di(ethoxyethyl)amine, N,N-di-(propoxyethyl)amine, and the like. Preferred secondary amines are N,N-dimethylamine, N,N-diethylamine and N,N-di-n-propylamine.

Cyclic secondary amines may also be employed to form the polyisobutenyl mono-amines or polyisobutenyl polyamines used in the instant invention. In such cyclic compounds, R¹ and R² of the formula hereinabove, when taken together, form one or more five- or six-membered rings containing up to about 20 carbon atoms. The ring containing the amine nitrogen atom is generally saturated, but may be fused to one or more saturated or unsaturated rings. The rings may be substituted with hydrocarbyl groups of from 1 to about 10 carbon atoms and may contain one or more oxygen atoms.

Suitable cyclic secondary amines include piperidine, 4-methylpiperidine, pyrrolidine, morpholine, 2,6-dimethylmorpholine, and the like.

The number average molecular weight M_{N} of the polyisobutyl group in the polyisobutyl monoamines, polyisobutyl polyamines and polyisobutyl-substituted Mannich adducts used in the instant invention as nitrogen-containing dispersant component (A) is in the range of from 650 to 1800 Dalton, preferably of from 700 to 1500 Dalton, most preferably of from 750 to 1300 Dalton. As already stated for the polyisobutene precursors, the polyisobutyl monoamines, polyisobutyl polyamines and polyisobutyl-substituted Mannich adducts are mostly not pure single products, but rather mixtures of compounds having number average molecular weights as indicated above. Usually, the range of molecular weights distribution will be relatively narrow having a maximum near the indicated molecular weight.

In an especially preferred embodiment, dispersant component (A) is a polyisobutyl monoamine with a number average molecular M_{N} weight of the polyisobutyl group of from 650 to 1800 Dalton, preferably of from 700 to 1500, most preferably of from 750 to 1300. The said polyisobutyl monoamine is preferably based on ammonia and/or preferably prepared by hydroformylation and subsequent reductive amination of corresponding highly reactive polyisobutenes, as described in EP-A 0 244 616. In more detail, highly reactive polyisobutenes having a high content of terminal vinylidene double bonds, especially at least 70 mol-%, more preferably at least 80 mol-%, of terminal vinylidene double bonds, are reacted with carbon monoxide and hydrogen in the presence of a hydroformylation catalyst, e.g. a suitable rhodium or cobalt catalyst, and preferably in an inert solvent such as a hydrocarbon solvent at a temperature in the range of typically from 80° C. to 200° C. and CO/H₂-pressures up to 600 bar. Thereafter, the oxo intermediate obtained is subjected to an reductive amination reaction in the presence of hydrogen, a suitable nitrogen compound, a suitable catalyst, e.g. Raney nickel or Raney cobalt, and preferably in an inert solvent such as a hydrocarbon solvent or an alcohol solvent at a temperature in the range of typically from 80° C. to 200° C. and H₂-pressures of from 80 to 300 bar.

Mannich adducts which are suitable as component (A) for the instant invention can be produced by reacting (i) 1 to 2 moles of at least one polyisobutylphenol which may carry at the aromatic ring system in addition to the polyisobutyl substituent with a number average molecular weight M_{N} of from 650 to 1800 Dalton, being derived preferably from highly reactive polyisobutene a as defined above, one or more, e.g. one, two or three, C₁ to C₇ alkyl substituents such as methyl, ethyl, n-propyl, iso-propyl, n-butyl, iso-butyl, sec.-butyl, tert.-butyl, n-pentyl or n-hexyl, with (ii) 1 to 3 moles of at least one C₁ to C₆ aldehyd such as formaldehyd, acetaldehyd and propionaldehyd which may be used in an oligomeric or polymeric form such as paraformaldehyd, and with (iii) 1 to 3 moles of at least one primary or secondary amine of formula HNR³R⁴ in which R³ denotes hydrogen, a C₁ to C₂₀ alkyl residue or a C₃ to C₂₀ cycloalkyl residue and R⁴ denotes a C₁ to C₂₀ alkyl residue or a C₃ to C₂₀ cycloalkyl residue, whereby both residues R³ and R⁴ can form together with the nitrogen atom they are attached to a ring system and/or can be independently from each other interrupted by one or more oxygen atoms and/or imino groups of formula —NR⁵— with R⁵ denoting hydrogen or a C₁ to C₄ alkyl group, and/or R⁴ can be terminated by a second —NH₂ group. Such Mannich adducts are known in the art, e.g. from WO 04/050806.

Examples for linear and branched primary amines of formula HNR³R⁴ are methylamine, ethylamine, n-propylamine, iso-propylamine, n-butylamine, iso-butylamine, sec.-butylamine, tert.-butylamine, n-pentylamine, n-hexylamine, n-heptylamine, n-octylamine, 2-ethylhexylamine, n-nonylamine, 3-propylheptylamine, n-decylamine, n-undecylamine, n-dodecylamine, n-tridecylamine, iso-tridecylamine, n-tetradecylamine, n-pentadecylamine, n-hexadecylamine, n-heptadecylamine, n-octadecylamine, n-nonadecylamine and n-eicosylamine.

Examples for linear, branched and cyclic secondary amines of formula HNR³R⁴ are dimethylamine, diethylamine, di n-propylamine, di-iso-propylamine, di-n-butylamine, di-iso-butylamine, di-sec.-butyl-amine, di-tert.-butylamine, di-n-pentylamine, d-in-hexylamine, di-n-heptylamine, di-n-octylamine, di-(2-ethylhexyl)amine, di-n-nonylamine, di-(3-propylheptyl)amine, di-n-decylamine, di-n-undecylamine, di-n-dodecylamine, di-n-tridecylamine, di-iso-tridecylamine, di-n-tetradecylamine, di-n-pentadecyl-amine, di-n-hexadecylamine, di-n-heptadecylamine, di-n-octadecylamine, di-n-nonadecylamine, di-n-eicosylamine, cyclooctylamine and cyclodecylamine.

Examples for amines of formula HNR³R⁴ which are interrupted by imino groups of formula —NR⁵— and/or can be terminated by a second —NH₂ group are N-(3,3-dimethylamino)propylamine, 1,2-ethylenediamine, 1,3-propylenediamine, 1,4-butylenediamine, diethylenetriamine, triethylenetetramine, tetraethylenepentamine and pentaethylenehexamine.

Typical examples for Mannich adducts suitable as component (A) are the reaction products of (i) 1 mole of 4-polyisobutylphenol (M_{N} of the polyisobutyl group=1000) with (ii) 1 mole of paraformaldehyd and (iii) 1 mole of dimethylamine or di-n-butylamine or di(2-ethylhexyl)amine. Further typical examples for Mannich adducts suitable as component (A) are the reaction products of (i) 2 moles of 4-polyisobutylphenol (M_{N} of the polyisobutyl group=1000) with (ii) 2 moles of paraformaldehyd and (iii) 1 mole of methylamine or n-butylamine or 2-ethylhexylamine or 3-(N,N-dimethylamino)propylamine.

The Carrier Oil (Component B)

The fuel-soluble, non-volatile carrier oil of component (B) is to be used as a necessary part of the fuel additive composition of the instant invention, in order to achieve the desired improvement in intake valve clean-up performance. The carrier oil is a chemically inert hydrocarbon-soluble liquid vehicle. The carrier oil of component (B) may be a synthetic oil or a mineral oil; for the instant invention, a refined petroleum oil is also understood to be a mineral oil.

Such carrier oils (also called carrier fluids) are believed to act as a carrier for the fuel additives and to assist in removing and retarding deposits. The carrier oil (B) may also exhibit synergistic deposit control and deposit removing properties when used in combination with components (A) and (C) of the instant fuel additive composition.

The carrier oil of component (B) is typically employed in amounts ranging from about 50 to about 2,000 ppm by weight of the gasoline fuel, preferably from 100 to 800 ppm of the gasoline fuel. Preferably, the ratio of carrier oil (B) to nitrogen-containing dispersant (A) in the fuel additive composition as well as in the gasoline fuel will range from 0.5:1 to 10:1, typically from 1:1 to 4:1.

When employed in fuel additive compositions or fuel additive concentrates, such as in the instant fuel additive composition, carrier oils will generally be present in amounts ranging from about 10 to about 60 weight percent, preferably from 20 to 40 weight percent (referring to the amount of all components in the composition or concentrate, respectively, including possible solvents).

Examples for suitable mineral carrier oils are in particular those of viscosity class Solvent Neutral (SN) 500 to 2000, as well as aromatic and paraffinic hydrocarbons and alkoxyalkanols. Another useful mineral carrier oil is a fraction known as “hydrocrack oil” which is obtained from refined mineral oil (boiling point of approximately 360 to 500° C.; obtainable from natural mineral oil which is isomerized, freed of paraffin components and catalytically hydrogenated under high pressure).

Examples for synthetic carrier oils which can be used for the instant invention are olefin polymers with a number average molecular weight of from 400 to 1800, based on polyalpha-olefins or poly-internal-olefins, especially those based on polybutene or on polyisobutene (hydrogenated or non-hydrogenated). Further examples for suitable synthetic carrier oils are polyesters, polyalkoxylates, polyethers, alkylphenol-initiated polyethers, and carboxylic acids of long-chain alkanols.

Examples for suitable polyethers which can be used for the instant invention are compounds containing polyoxy-C₂-C₄alkylene groups, especially polyoxy-C₃-C₄-alkylene groups, which can be obtained by reacting C₁-C₃₀-alkanols, C₂-C₆₀-alkandiols, C₁-C₃₀-alkylcyclohexanols or C₁-C₃₀-alkylphenols with 1 to 30 mol ethylene oxide and/or propylene oxide and/or butylene oxides per hydroxyl group, especially with 1 to 30 mol propylene oxide and/or butylene oxides per hydroxyl group. This type of compounds is described, for example, in EP-A 310 875, EP-A 356 725, EP-A 700 985 and U.S. Pat. No. 4,877,416.

Typical examples for suitable polyethers are tridecanol butoxylates, isotridecanol butoxylates, isononylphenol butoxylates, polyisobutenol butoxylates and polyisobutenol propoxylates.

Hydrocarbyl-terminated poly(oxyalkylene) polymers which may be employed in the present invention as component (B), are monohydroxy compounds, i.e., alcohols, and are often termed monohydroxy polyethers, or polyalkylene glycol monohydrocarbylethers, or “capped” poly(oxyalkylene).

The hydrocarbyl-terminated poly(oxyalkylene) alcohols may be produced by the addition of lower alkylene oxides, such as ethylene oxide, propylene oxide, the butylene oxides, or the pentylene oxides to the hydroxy compound under polymerization conditions. Methods of production and properties of these polymers are disclosed in U.S. Pat. Nos. 2,841,479 and 2,782,240 and Kirk-Othmer's “Encyclopedia of Chemical Technology”, 2nd Ed. Volume 19, p. 507. In the polymerization reaction, a single type of alkylene oxide may be employed, e.g., propylene oxide, in which case the product is a homopolymer, e.g., a poly(oxyalkylene) propanol. However, copolymers are equally satisfactory and random copolymers are readily prepared by contacting the hydroxyl-containing compound with a mixture of alkylene oxides, such as a mixture of propylene and butylene oxides. Block copolymers of oxyalkylene units also provide satisfactory poly(oxyalkylene) polymers for the practice of the present invention. Random polymers are more easily prepared when the reactivities of the oxides are relatively equal. In certain cases, when ethylene oxide is copolymerized with other oxides, the higher reaction rate of ethylene oxide makes the preparation of random copolymers difficult. In either case, block copolymers can be prepared. Block copolymers are prepared by contacting the hydroxyl-containing compound with first one alkylene oxide, then the others in any order, or repetitively, under polymerization conditions. A particular block copolymer is represented by a polymer prepared by polymerizing propylene oxide on a suitable monohydroxy compound to form a poly(oxypropylene) alcohol and then polymerizing butylene oxide on the poly(oxyalkylene) alcohol.

In general, the poly(oxyalkylene) polymers are mixtures of compounds that differ in polymer chain length. However, their properties closely approximate those of the polymer represented by the average composition and molecular weight.

Examples of carboxylic esters of long-chain alkanols are esters of mono-, di- and tricarboxylic acids with long-chain alkanols or polyhydric alcohols such as described e.g. in DE-A 38 38 918. Suitable mono-, di- and tricarboxylic acids are aliphatic or aromatic carboxylic acids. Suitable alkanols and polyhydric alcohols contain 6 to 24 carbon atoms. Typical examples of such esters are the adipates, phthalates, iso-phthalates, terephthalates and trimellitates of isooctanol, isononanol, isodecanol and isotridecanol, e.g. di-n-tridecyl phthalate or di-iso-tridecyl phthalate.

Examples for particularly useful synthetic carrier oils are alcohol-initiated polyethers containing about 5 to 35, e.g. 5 to 30 C₃-C₆-alkylenoxide units, such as propylenoxide, n-butylenoxide and iso-butylenoxide units or mixtures thereof. Non-limiting examples for alcoholic starters are long-chain alkanols or phenols substituted by long-chain alkyl groups, where the alkyl group preferably is linear or branched C₆-C₁₈-alkyl. Preferred examples for the alcoholic starters are tridecanol and nonylphenol.

Further suitable synthetic carrier oils are alkoxylated alkylphenols, such as described e.g. in DE-A 10 102 913.

Preferably, synthetic carrier oils are used. Preferred synthetic carrier oils are alkanol alkoxylates, in particular alkanol propoxylates and alkanol butoxylates.

In an especially preferred embodiment, carrier oil component (B) comprises at least one polyether obtained from C₁-C₃₀-alkanols, especially C₆-C₁₈-alkanols, or C₂-C₆₀-alkandiols, especially C₈-C₂₄-alkandiols, and from 1 to 30 mol, especially 5 to 30 mol, in sum, of propylene oxide and/or butylene oxides. Other synthetic carrier oils and/or mineral carrier oils may be present in component (B) in minor amounts.

The Dispersant Booster (Component C)

The polyisobutenes which are suitable for preparing the low-molecular weight polyisobutyl-substituted amine dispersant boosters used in the present invention as component (C) can include polyisobutenes which comprise at least about 20%, preferably at least 50%, more preferably at least 70 mol-%, most preferably at least 80 mol-%, of the more reactive methylvinylidene isomer (i.e. with the terminal vinylidene double bond). Suitable polyisobutenes include those prepared using BF₃ catalysts. The preparation of such polyisobutenes in which the methylvinylidene isomer comprises a high percentage of the total composition is for example described in U.S. Pat. No. 4,152,499 and U.S. Pat. No. 4,605,808, starting either from pure isobutene or from technical C₄ streams containing high percentages of isobutene such as raffinate I.

Furthermore, such polyisobutene suitable for preparing the low-molecular weight polyisobutyl-substituted amine dispersant boosters used in the present invention as component (C) can bei oligomers of isobutene, e.g. triisobutene, tetraisobutene, pentaisobutene, hexaisobutene, heptaisobutene, octaisobutene, nonaisobutene, decaisobutene, undecaisobutene, dodecaisobutene or mixtures thereof.

In most instances, the above polyisobutene precursors are not a pure single product, but rather a mixture of compounds having an number average molecular weight in the above range of 200 to 650 Dalton. Usually, the range of molecular weights distribution will be relatively narrow having a maximum near the indicated molecular weight.

The amine component of the said low-molecular weight polyisobutyl monoamines or polyisobutyl polyamines, respectively, may be derived from ammonia, a monoamine or a polyamine.

The monoamine or polyamine component comprises amines having from 1 to about 12 amine nitrogen atoms and from 1 to 40 carbon atoms. The carbon to nitrogen ratio may be between about 1:1 and about 10:1. Generally, the monoamine will contain from 1 to about 40 carbon atoms and the polyamine will contain from 2 to about 12 amine nitrogen atoms and from 2 to about 40 carbon atoms.

The amine component may be a pure single product or a mixture of compounds having a major quantity of the designated amine.

When the amine component is a polyamine, it will preferably be a polyalkylene polyamine, including alkylene diamine. Preferably, the alkylene group will contain from 2 to 6 carbon atoms, more preferably from 2, 3 or 4 carbon atoms. Examples of such polyamines include ethylene diamine, diethylene triamine, triethylene tetramine, tetraethylene pentamine and pentaethylene hexamine. Preferred polyamines are ethylene diamine and diethylene triamine.

Particularly preferred polyisobutyl polyamines include polyisobutyl ethylene diamine and polyisobutyl diethylene triamine. The polyisobutyl group is substantially saturated.

The polyisobutyl monoamines or polyisobutyl polyamines employed in the fuel additive composition of the instant invention as component (C) are prepared by conventional procedures known in the art, especially by hydroformylation and subsequent reductive amination of corresponding highly reactive polyisobutenes, e.g. in analogy to the teachings of EP-A 0 244 616. In more detail, highly reactive polyisobutenes having a high content of terminal vinylidene double bonds, especially at least 70 mol-%, more preferably at least 80 mol-%, of terminal vinylidene double bonds, are reacted with carbon monoxide and hydrogen in the presence of a hydroformylation catalyst, e.g. a suitable rhodium or cobalt catalyst, and preferably in an inert solvent such as a hydrocarbon solvent at a temperature in the range of typically from 80° C. to 200° C. and CO/H₂-pressures up to 600 bar. Thereafter, the oxo intermediate obtained is subjected to an reductive amination reaction in the presence of hydrogen, a suitable nitrogen compound, a suitable catalyst, e.g. Raney nickel or Raney cobalt, and preferably in an inert solvent such as a hydrocarbon solvent or an alcohol solvent at a temperature in the range of typically from 80° C. to 200° C. and H₂-pressures of from 80 to 300 bar.

The amine portion of the molecule may carry one or more substituents. Thus, the carbon and/or, in particular, the nitrogen atoms of the amine may carry substituents selected from hydrocarbyl groups of from 1 to about 10 carbon atoms, acyl groups of from 2 to about 10 carbon atoms, and monoketo, monohydroxy, mononitro, monocyano, lower alkyl and lower alkoxy derivatives thereof. “Lower” as used herein means a group containing from 1 to about 6 carbon atoms. At least one of the hydrogen atoms on one of the basic nitrogen atoms of the polyamine may not be substituted so that at least one of the basic nitrogen atoms of the polyamine is a primary or secondary amino nitrogen atom.

A polyamine finding use within the scope of the present invention as amine component for the polyisobutyl polyamines may be a polyalkylene polyamine, including substituted polyamines, e.g., alkyl and hydroxyalkyl-substituted polyalkylene polyamine. Among the polyalkylene polyamines, those containing 2 to 12 amino nitrogen atoms and 2 to 24 carbon atoms should be mentioned, in particular C₂-C₃ alkylene polyamines. Preferably, the alkylene group contains from 2 to 6 carbon atoms, there being preferably from 2 to 3 carbon atoms between the nitrogen atoms. Such groups are exemplified by ethylene, 1,2-propylene, 2,2-dimethylpropylene, trimethylene, 1,3-(2-hydroxy)propylene.

Examples of such polyamines include ethylene diamine, diethylene triamine, di(trimethylene) triamine, 1,2-propylene diamine, 1,3-propylene diamine, dipropylene triamine, triethylene tetraamine, tripropylene tetraamine, tetraethylene pentamine, pentaethylene hexamine. hexamethylene diamine, and 3-(N,N-dimethylamino) propylamine. Such amines encompass isomers such as branched-chain polyamines and previously-mentioned substituted polyamines, including hydroxy- and hydrocarbyl-substituted polyamines.

The amine component for the polyisobutyl monoamines or polyisobutyl polyamines also may be derived from heterocyclic polyamines, heterocyclic substituted amines and substituted heterocyclic compounds, wherein the heterocycle comprises one or more five- or six-membered rings containing oxygen and/or nitrogen. Such heterocyclic rings may be saturated or unsaturated and substituted with groups as defined above.

As examples of heterocyclic compounds there may be mentioned 2-methylpiperazine, N-(2-hydroxyethyl)-piperazine, 1,2-bis-(N-piperazinyl)ethane, N,N′-bis(N-piperazinyl)piperazine, 2-methylimidazoline, 3-aminopiperidine, 3-aminopyridine, N-(3-aminopropyl)-morpholine, N-(beta-aminoethyl)piperazine, N-(betaaminoethyl)piperidine, 3-amino-N-ethylpiperidine, N-(betaaminoethyl) morpholine, N,N′-di(beta-aminoethyl)piperazine, N,N′-di(beta-aminoethyl)imidazolidone-2,1,3-dimethyl-5(beta-aminoethyl)hexahydrotriazine, N-(betaaminoethyl)-hexahydrotriazine, 5-(beta-aminoethyl)-1,3,5-dioxazine.

Alternatively, the amine component for the polyisobutyl monoamines may be derived from a monoamine having the formula HNR¹R² wherein R¹ and R² are independently selected from the group consisting of hydrogen and hydrocarbyl of 1 to about 20 carbon atoms and, when taken together, R¹ and R² may form one or more five- or six-membered rings containing up to about 20 carbon atoms. Preferably, R¹ is hydrogen and R² is a hydrocarbyl group having 1 to about 10 carbon atoms. More preferably, R¹ and R² are hydrogen. The hydrocarbyl groups may be straight-chain or branched and may be aliphatic, alicyclic, aromatic or combinations thereof. The hydrocarbyl groups may also contain one or more oxygen atoms.

Typical primary amines are exemplified by N-methylamine, N-ethylamine, N-n-propylamine, N-isopropylamine, N-n-butylamine, N-isobutylamine, N-sec.-butylamine, N-tert.-butylamine, N-n-pentylamine, N-cyclopentylamine, N-n-hexylamine, N-cyclohexylamine, N-octylamine, N-decylamine, N-dodecylamine, N-octadecylamine, N-benzylamine, N-(2-phenylethyl)amine, 2-aminoethanol, 3-amino-1-proponal, 2-(2-aminoethoxy)ethanol, N-(2-methoxyethyl)amine, N-(2-ethoxyethyl)amine, and the like. Preferred primary amines are N-methylamine, N-ethylamine and N-n-propylamine.

Typical secondary amines include N,N-dimethylamine, N,N-diethylamine, N,N-di-n-propylamine, N,N-diisopropylamine, N,N-di-n-butylamine, N,N-di-sec-butylamine, N,N-di-n-pentylamine, N,N-di-n-hexylamine, N,N-dicyclohexylamine, N,N-dioctylamine, N-ethyl-N-methylamine, N-methyl-N-n-propylamine, N-n-butyl-N-methylamine, N-methyl-N-octylamine, N-ethyl-N-isopropylamine, N-ethyl-N-octylamine, N,N-di-(2-hydroxyethyl)amine, N,N-di(3-hydroxypropyl)amine, N,N-di(ethoxyethyl)amine, N,N-di-(propoxyethyl)amine, and the like. Preferred secondary amines are N,N-dimethylamine, N,N-diethylamine and N,N-di-n-propylamine.

Cyclic secondary amines may also be employed to form the polyisobutenyl monoamines or polyisobutenyl polyamines used in the instant invention. In such cyclic compounds, R¹ and R² of the formula hereinabove, when taken together, form one or more five- or six-membered rings containing up to about 20 carbon atoms. The ring containing the amine nitrogen atom is generally saturated, but may be fused to one or more saturated or unsaturated rings. The rings may be substituted with hydrocarbyl groups of from 1 to about 10 carbon atoms and may contain one or more oxygen atoms.

Suitable cyclic secondary amines include piperidine, 4-methylpiperidine, pyrrolidine, morpholine, 2,6-dimethylmorpholine, and the like.

The number average molecular weight M_{N} of the polyisobutyl group in the polyisobutyl monoamines, polyisobutyl polyamines and polyisobutyl-substituted Mannich adducts used in the instant invention as dispersant booster component (C) is in the range of from 200 to 650 Dalton, preferably of from 250 to 600 Dalton, most preferably of from 300 to 550 Dalton. As already stated for the polyisobutene precursors, the polyisobutyl monoamines, polyisobutyl polyamines and polyisobutyl-substituted Mannich adducts are mostly not pure single products, but rather mixtures of compounds having number average molecular weights as indicated above. Usually, the range of molecular weights distribution will be relatively narrow having a maximum near the indicated molecular weight.

In an especially preferred embodiment, dispersant booster component (C) is a polyisobutyl monoamine with a number average molecular weight M_{N} of the polyisobutyl group of from 200 to 650 Dalton, preferably of from 250 to 600 Dalton, most preferably of from 300 to 550. The said polyisobutyl monoamine is preferably based on ammonia and/or preferably prepared by hydroformylation and subsequent reductive amination of corresponding highly reactive polyisobutenes, as described in EP-A 0 244 616. In more detail, highly reactive polyisobutenes having a high content of terminal vinylidene double bonds, especially at least 70 mol-%, more preferably at least 80 mol-%, of terminal vinylidene double bonds, are reacted with carbon monoxide and hydrogen in the presence of a hydroformylation catalyst, e.g. a suitable rhodium or cobalt catalyst, and preferably in an inert solvent such as a hydrocarbon solvent at a temperature in the range of typically from 80° C. to 200° C. and CO/H₂-pressures up to 600 bar. Thereafter, the oxo intermediate obtained is subjected to an reductive amination reaction in the presence of hydrogen, a suitable nitrogen compound, a suitable catalyst, e.g. Raney nickel or Raney cobalt, and preferably in an inert solvent such as a hydrocarbon solvent or an alcohol solvent at a temperature in the range of typically from 80° C. to 200° C. and H₂-pressures of from 80 to 300 bar.

Mannich adducts which are suitable as component (C) for the instant invention can be produced by reacting (i) 1 to 2 moles of at least one polyisobutylphenol which may carry at the aromatic ring system in addition to the polyisobutyl substituent with a number average molecular weight M_{N} of from 200 to 650 Dalton, being derived preferably from highly reactive polyisobutene a as defined above, one or more, e.g. one, two or three, C₁ to C₇ alkyl substituents such as methyl, ethyl, n-propyl, iso-propyl, n-butyl, iso-butyl, sec.-butyl, tert.-butyl, n-pentyl or n-hexyl, with (ii) 1 to 3 moles of at least one C₁ to C₆ aldehyd such as formaldehyd, acetaldehyd and propionaldehyd which may be used in an oligomeric or polymeric form such as paraformaldehyd, and with (iii) 1 to 3 moles of at least one primary or secondary amine of formula HNR³R⁴ in which R³ denotes hydrogen, a C₁ to C₂₀ alkyl residue or a C₃ to C₂₀ cycloalkyl residue and R⁴ denotes a C₁ to C₂₀ alkyl residue or a C₃ to C₂₀ cycloalkyl residue, whereby both residues R³ and R⁴ can form together with the nitrogen atom they are attached to a ring system and/or can be independently from each other interrupted by one or more oxygen atoms and/or imino groups of formula —NR⁵— with R⁵ denoting hydrogen or a C₁ to C₄ alkyl group, and/or R² can be terminated by a second —NH₂ group. Such Mannich adducts are known in the art, e.g. from WO 04/050806.

Examples for linear and branched primary amines of formula HNR³R⁴ are methylamine, ethylamine, n-propylamine, iso-propylamine, n-butylamine, iso-butylamine, sec.-butylamine, tert.-butylamine, n-pentylamine, n-hexylamine, n-heptylamine, n-octylamine, 2-ethylhexylamine, n-nonylamine, 3-propylheptylamine, n-decylamine, n-undecylamine, n-dodecylamine, n-tridecylamine, iso-tridecylamine, n-tetradecylamine, n-pentadecylamine, n-hexadecylamine, n-heptadecylamine, n-octadecylamine, n-nonadecylamine and n-eicosylamine.

Examples for linear, branched and cyclic secondary amines of formula HNR³R⁴ are dimethylamine, diethylamine, di n-propylamine, di-iso-propylamine, di-n-butylamine, di-iso-butylamine, di-sec.-butyl-amine, di-tert.-butylamine, di-n-pentylamine, d-in-hexylamine, di-n-heptylamine, di-n-octylamine, di-(2-ethylhexyl)amine, di-n-nonylamine, di-(3-propylheptyl)amine, di-n-decylamine, di-n-undecylamine, di-n-dodecylamine, di-n-tridecylamine, di-iso-tridecylamine, di-n-tetradecylamine, di-n-pentadecyl-amine, di-n-hexadecylamine, di-n-heptadecylamine, di-n-octadecylamine, di-n-nonadecylamine, di-n-eicosylamine, cyclooctylamine and cyclodecylamine.

Examples for amines of formula HNR³R⁴ which are interrupted by imino groups of formula —NR⁵— and/or can be terminated by a second —NH₂ group are N-(3,3-dimethylamino)propylamine, 1,2-ethylenediamine, 1,3-propylenediamine, 1,4-butylenediamine, diethylenetriamine, triethylenetetramine, tetraethylenepentamine and pentaethylenehexamine.

Typical examples for Mannich adducts suitable as component (A) are the reaction products of (i) 1 mole of 4-polyisobutylphenol (M_{N} of the polyisobutyl group=420) with (ii) 1 mole of paraformaldehyd and (iii) 1 mole of dimethylamine or di-n-butylamine or di(2-ethylhexyl)amine. Further typical examples for Mannich adducts suitable as component (A) are the reaction products of (i) 2 moles of 4-polyisobutylphenol (M_{N} of the polyisobutyl group=420) with (ii) 2 moles of paraformaldehyd and (iii) 1 mole of methylamine or n-butylamine or 2-ethylhexylamine or 3-(N,N-dimethylamino)propylamine.

In the fuel additive composition of the instant invention, dispersant component (A) can be a polyisobutyl monoamine, a polyisobutyl polyamine, a Mannich adduct of polyisobutylphenols, aldehyds and monoamines or a Mannich adduct of polyisobutylphenols, aldehyds and polyamines or a mixture of the aforementioned dispersant types in combination with (C) a low-molecular weight polyisobutyl monoamine.

Furthermore, in the fuel additive composition of the instant invention, dispersant component (A) can be a polyisobutyl monoamine, a polyisobutyl polyamine, a Mannich adduct of polyisobutylphenols, aldehyds and monoamines or a Mannich adduct of polyisobutylphenols, aldehyds and polyamines or a mixture of the aforementioned dispersant types in combination with (C) a low-molecular weight polyisobutyl polyamine.

Furthermore, in the fuel additive composition of the instant invention, dispersant component (A) can be a polyisobutyl monoamine, a polyisobutyl polyamine, a Mannich adduct of polyisobutylphenols, aldehyds and monoamines or a Mannich adduct of polyisobutylphenols, aldehyds and polyamines or a mixture of the aforementioned dispersant types in combination with (C) a Mannich adduct of a low-molecular weight polyisobutylphenol, an aldehyd and a monoamine.

Furthermore, in the fuel additive composition of the instant invention, dispersant component (A) can be a polyisobutyl monoamine, a polyisobutyl polyamine, a Mannich adduct of polyisobutylphenols, aldehyds and monoamines or a Mannich adduct of polyisobutylphenols, aldehyds and polyamines or a mixture of the aforementioned dispersant types in combination with (C) a Mannich adduct of a low-molecular weight polyisobutylphenol, an aldehyd and a polyamine.

In a preferred embodiment, dispersant component (A) and detergent booster component (C) comprise solely polyisobutyl monoamine or polyisobutyl polyamine species with the same monoamine or polyamine end groups. The said end group is preferably the NH₂ group derived from ammonia.

In case of the same amine end groups for the polyisobutyl amines, dispersant component (A) and detergent booster component (C) typically form a mixture of homologue polyisobutyl amines exhibiting a bimodal molecular weight distribution. The same is true for a mixture of Mannich adducts with the same methyleneamino end groups both for components (A) and (C) based on homologue polyisobutylphenols. A bimodal molecular weight distribution is normally characterized for organic polymers by an asymmetric graph (peak) in the fraction versus molecular weight plot from an analytical method like gel permeation chromatography (“GPC”), as used for determination of the instant M_{N} values. Small differences in molecular weight result in a shoulder of the peak; with growing differences the shoulder forms a second peak. The said situation could also be described in mathematical terms as follows: the first deviation of the graph exhibits two maxima, whereas the first deviation of a monomodal graph only exhibits one maximum and one minimum. In case of bimodal molecular weight distribution, components (A) and (C) can be produced from the same polymerization and subsequent amination reaction of isobutene or by the same production of polyisobutylphenols from isobutene, respectively, with or without separation of the two species differing in number average molecular weight e.g. by means of chromatography or fractional distillation; the said separation can be done before or after the amination step or the Mannich addition reaction, respectively. Alternatively, components (A) and (C) can be produced separately and only mixed thereafter together with component (B).

The Fuel Additive Composition

The instant fuel additive composition may be formulated as a concentrate, using an inert stable oleophilic (i.e., dissolves in fuel) organic solvent boiling in the range of about 65° C. to 205° C. Preferably, an aliphatic or an aromatic hydrocarbon solvent is used, such as benzene, toluene, xylene or higher-boiling aromatics or aromatic thinners. Aliphatic alcohols of about 3 to 8 carbon atoms, such as isopropanol, isobutylcarbinol, n-butanol, 2-ethylhexanol, and the like, in combination with hydrocarbon solvents, are also suitable for use in such concentrate. In the concentrate, the amount of the instant fuel additive composition will be ordinarily at least 10% by weight to about 90% by weight, as for example 40 to 85 weight percent or 50 to 80 weight percent.

In gasoline fuels, other fuel additives may be employed with the additives of the present invention, including, for example, oxygenates, such as tert.-butyl methyl ether, antiknock agents, such as methylcyclopentadienyl manganese tricarbonyl, and other dispersants/detergents, such as various hydrocarbyl amines, succinimides or polyetheramines, i.e. hydrocarbyl poly(oxyalkylene) amines. A list of suitable other dispersant/detergent additives is for example given in WO 00/47698 or in EP-A 1 155 102.

Also included may be lead scavengers, such as aryl halides, e.g., dichlorobenzene, or alkyl halides, e.g., ethylene dibromide. In addition, antioxidants, metal deactivators, pour point depressants, corrosion inhibitors and demulsifiers may be present.

In an especially preferred embodiment, the weight ratio of dispersant component (A) to dispersant booster component (C) is in the range of from 0.1:1 to 10:1, especially of from 0.3:1 to 7:1, thus provided the best improvement of intake valve clean-up performance of gasoline fuels.

An interaction between all three components (A), (B) and (C) is necessary to achieve the desired improvement in intake valve clean-up performance. In the instant fuel additive composition, the dispersant booster component (C) may exhibit a synergistic effect in this respect when used in combination with components (A) and (B) of the instant fuel additive composition.

The Fuel Composition

The fuel additive composition of the present invention will generally be employed in a liquid hydrocarbon distillate fuel boiling in the gasoline range. It is in principle suitable for use in all types of gasoline, including “light” and “severe” gasoline species. The gasoline fuels may also contain amounts of other fuel components such as, for example, ethanol.

The proper concentration of the instant fuel additive composition necessary in order to achieve the desired intake valve clean-up performance varies depending upon the type of fuel employed, and may also be influenced by the presence of other detergents, dispersants and other additives, etc. Generally, however, from 80 to 8000 weight ppm, especially from 180 to 2600 weight ppm, of the instant fuel additive composition per part of base fuel is needed to achieve the best results.

In an especially preferred embodiment, dispersant component (A) is present in the instant fuel composition at a level of from more than 20 to 3000 ppm, especially from 70 to 800 ppm, carrier oil component (B) at a level of from 50 to 2000 ppm, especially from 100 to 600 ppm, and amine component (C) at a level of from 10 to 3000 ppm, especially from 30 to 1200 ppm (all ppm values refer to the weight).

Typically, gasoline fuels, which may be used according to the present invention exhibit, in addition, one or more of the following features:

The aromatics content of the gasoline is preferably not more than 50 volume % and more preferably not more than 45 volume %. Preferred ranges for the aromatics content are from 1 to 45 volume % and particularly from 5 to 40 volume %.

The sulfur content of the gasoline is preferably not more than 100 ppm by weight and more preferably not more than 50 ppm by weight. Preferred ranges for the sulfur content are from 0.5 to 150 ppm by weight and particularly from 1 to 100 ppm by weight.

The gasoline has an olefin content of not more than 21 volume %, preferably not more than 18 volume %, and more preferably not more than 10 volume %. Preferred ranges for the olefin content are from 0.1 to 21 volume % and particularly from 2 to 18 volume %.

The gasoline has a benzene content of not more than 1.0 volume % and preferably not more than 0.9 volume %. Preferred ranges for the benzene content are from 0 to 1.0 volume % and preferably from 0.05 to 0.9 volume %.

The gasoline has an oxygen content of not more than 45 weight %, preferably from 0 to 45 weight %, and most preferably from 0.1 to 2.7 weight % (first type) or most preferably from 2.7 to 45 weight % (second type). The gasoline of the second type mentioned above is a mixture of lower alcohols such as methanol or especially ethanol, which derive preferably from natural source like plants, with mineral oil based gasoline, i.e. usual gasoline produced from crude oil. An example for such gasoline is “E 85”, a mixture of 85 volume % of ethanol with 15 volume % of mineral oil based gasoline.

The content of alcohols, especially lower alcohols, and ethers in a gasoline of the first type mentioned in the above paragraph is normally relatively low. Typical maximum contents are for methanol 3 volume %, for ethanol 5 volume %, for isopropanol 10 volume %, for tert.-butanol 7 volume %, for isobutanol 10 volume %, and for ethers containing 5 or more carbon atoms in the molecule 15 volume %.

For example, a gasoline which has an aromatics content of not more than 38 volume % and at the same time an olefin content of not more than 21 volume %, a sulfur content of not more than 50 ppm by weight, a benzene content of not more than 1.0 volume % and an oxygen content of from 0.1 to 2.7 weight % may be applied.

The summer vapor pressure of the gasoline is usually not more than 70 kPa and preferably not more than 60 kPa (at 37° C.).

The research octane number (“RON”) of the gasoline is usually from 90 to 100. A usual range for the corresponding motor octane number (“MON”) is from 80 to 90.

The above characteristics are determined by conventional methods (DIN EN 228).

The Internal Combustion Engine

The above dispersant booster component (C) is preferably used as an intake valve clean-up booster in accordance with the instant invention in gasoline-operated port fuel injection internal combustion engines which are different in view of their construction and their mode of operation from direct injection spark ignition engines.

Experimental Part

The following examples are presented to illustrate specific embodiments of this invention and are not to be construed in any way as limiting the scope of the invention.

### EXAMPLES 1 AND 2

### Determination of Intake Valve Deposits (“IVD”)

Intake valve deposits were determined in gasoline-operated internal combustion engines of the Mercedes Benz M 102E type according to test procedure CEC F-05-A-93 (Examples 1a and 1b) and of the Mercedes Benz M 111 type according to test procedure CEC F-20-A-98 (Examples 2a and 2b). A usual Eurosuper gasoline according to EN 228 was used as the base fuel. The deposits on the four valves of the engines were determined and the average thereof value was calculated.

The following additives were used:

A1: polyisobutyl monoamine based on highly reactive polyisobutene with a methylvinylidene content of 80 mole-% and M_{N}=1000 subjected to hydroformylation and subsequent reductive amination with ammonia

B1: polyether carrier oil obtained from tridecanol and 22 moles of butylene oxide

C1: polyisobutyl monoamine based on highly reactive polyisobutene with a methylvinylidene content of 80 mole-% and M_{N}=420 subjected to hydroformylation and subsequent reductive amination with ammonia

### Example 1a

### For Comparison

A Mercedes Benz M 102E engine was run according to CEC F-05-A-93 for 60 hours with an Eurosuper gasoline fuel containing 300 wt.-ppm of A1 and 75 wt.-ppm of B1. As a result, the following IVD values were obtained: 12 mg, 20 mg, 67 mg, 8 mg; average: 21 mg. Running the same test without any additives resulted in an average IVD value of 153 mg.

Example 1b

### According to the Instant Invention

The same Mercedes Benz M 102E engine was run according to CEC F-05-A-93 for 60 hours with an Eurosuper gasoline fuel containing 300 wt.-ppm of A1, 75 wt.-ppm of B1 and 50 mg of C1. As a result, the following IVD values were obtained: 3 mg, 0 mg, 12 mg, 10 mg; average: 6 mg.

### Example 2a

### For Comparison

A Mercedes Benz M 111 engine was run according to CEC F-20-A-98 for 60 hours with an Eurosuper gasoline fuel containing 400 wt.-ppm of A1 and 100 wt.-ppm of B1. As a result, the following IVD values were obtained (double determination): 143/175 mg, 73/164 mg, 55/68 mg, 156/148 mg; average: 123 mg. Running the same test without any additives resulted in an average IVD value of 359 mg.

Example 2b

### According to the Instant Invention

The same Mercedes Benz M 111 engine was run according to CEC F-20-A-98 for 60 hours with an Eurosuper gasoline fuel containing 300 wt.-ppm of A1, 75 wt.-ppm of B1 and 50 mg of C1. As a result, the following IVD values were obtained: 80/87 mg, 46/42 mg, 73/48 mg, 125/135 mg; average: 80 mg.

1. A fuel additive composition comprising:
(A) at least one nitrogen-containing dispersant selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 650 to 1800 Dalton,
(B) at least one carrier oil which is substantially free of nitrogen, selected from synthetic carrier oils and mineral carrier oils, and
(C) at least one dispersant booster selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 200 to 650 Dalton,
with the proviso that the difference between the M_{N} of the polyisobutyl group of component (A) and the M_{N} of the polyisobutyl group of component (C) is more than 100 Dalton.

2. The fuel additive composition according to claim 1, wherein dispersant booster component (C) comprises at least one polyisobutyl monoamine with a number average molecular weight M_{N} of the polyisobutyl group of from 250 to 600 Dalton.

3. The fuel additive composition according to claim 1 or 2, wherein dispersant component (A) comprises at least one polyisobutyl monoamine with a number average molecular weight M_{N} of the polyisobutyl group of from 700 to 1500 Dalton.

4. The fuel additive composition according to claims 1 to 3, wherein dispersant component (A) and detergent booster component (C) comprise solely polyisobutyl monoamine or polyisobutyl polyamine species with the same monoamine or polyamine end groups.

5. The fuel additive composition according to claims 2 to 4, wherein dispersant component (A) and/or detergent booster component (C) are prepared by hydroformylation and subsequent reductive amination of corresponding highly reactive polyisobutenes.

6. The fuel additive composition according to claims 1 to 5, wherein the weight ratio of dispersant component (A) to detergent booster component (C) is in the range of from 0.1:1 to 10:1.

7. The fuel additive composition according to claims 1 to 6, wherein carrier oil component (B) comprises at least one polyether obtained from C₁-C₃₀-alkanols or C₂-C₆₀-alkandiols and from 1 to 30 mol, in sum, of ethylene oxide and/or propylene oxide and/or butylene oxides.

8. A fuel composition comprising a major amount of a liquid fuel in gasoline boiling range and a minor amount of a fuel additive composition according to claims 1 to 7.

9. The fuel composition according to claims 8, wherein dispersant component (A) is present at a level of from 20 to 3000 ppm, carrier oil component (B) at a level of from 50 to 2000 ppm, and dispersant booster component (C) at a level of from 10 to 3000 ppm.

10. The use of a polyisobutyl monoamine, a polyisobutyl polyamine, a Mannich adduct of a polyisobutylphenol, an aldehyd and a monoamine or a Mannich adduct of a polyisobutylphenol, an aldehyd and a polyamine (C), each with a number average molecular weight M_{N} of the polyisobutyl group of from 200 to 650 Dalton, as set out in claim 1, as a dispersant booster in internal combustion engines operated with a liquid fuel in the gasoline boiling range containing minor amounts of (A) at least one nitrogen-containing dispersant selected from polyisobutyl monoamines, polyisobutyl polyamines, Mannich adducts of polyisobutylphenols, aldehyds and monoamines and Mannich adducts of polyisobutylphenols, aldehyds and polyamines, each with a number average molecular weight M_{N} of the polyisobutyl group of from 650 to 1800 Dalton, and (B) at least one carrier oil which is substantially free of nitrogen, selected from synthetic carrier oils and mineral carrier oils.

11. The use of a low-molecular weight polyisobutyl monoamine, polyisobutyl polyamine, Mannich adduct of a polyisobutylphenol, an aldehyd and a monoamine or Mannich adduct of a polyisobutylphenol, an aldehyd and a polyamine (C), according to claim 10, as an intake valve clean-up booster in gasoline-operated port fuel injection internal combustion engines.
